(19) United States
(12) Patent Application Publication
(10) Pub. No.: US 2006/0251602 A1
(43) Pub. Date: Nov. 9, 2006

(54) Hair treatment agents with surfactant mixtures

(76) Inventors: Dieter Goddinger, Klein Nordende, DE (US); Dirk Hentrich, Hamburg (DE)

Correspondence Address: DANN DORFMAN HERRELL AND SKILLMAN;A PROFESSIONAL CORPORATION, 1601 MARKET STREET, SUITE 2400, PHILADELPHIA, PA, 19103-2307, US

(21) Appl. No.: 11/208,206

(22) Filed: Aug. 19, 2005

Related U.S. Application Data
(63) Continuation of application No. PCT/EP04/01264, filed on Feb. 11, 2004.

(30) Foreign Application Priority Data
Feb. 19, 2003 (DE) DE 103 07 115.6

(51) Int. Cl. A61K 8/73; A61K 8/97

(52) U.S. Cl. 424/70.13; 424/74

(57) ABSTRACT

The invention relates to a composition useful as a cosmetic hair treatment agent based on a) a surfactant mixture containing: A) at least one anionic surfactant; B) at least one amphoteric surfactant, and; C) at least one additional surfactant selected from the group consisting of acyl glutamates, amino oxides and of alkyl polyglucosides. The surfactant mixture also contains: b) at least one additional hair care substance selected from the group consisting of fatty alcohols, cationic surfactants, cationic polymers, cationically derivatized protein hydrolysates, water-insoluble volatile silicones and/or of water-soluble volatilized silicones and vitamins and/or provitamins and/or physiologically compatible derivatives thereof. This agent can be excellently formulated as a 2-in-1 shampoo that, while simultaneously cleaning and caring for, also positively influences the volume and body of the hair.

### CROSS-REFERENCE TO RELATED APPLICATIONS

This application is a continuation application under 35 U.S.C. §365(c) and 35 U.S.C. §120 of international application PCT/EP2004/001264, filed Feb. 11, 2004. This application also claims priority under 35 U.S.C. §119 of DE 103 07 115.6, filed Feb. 19, 2003, which is incorporated herein by reference in its entirety.

### STATEMENT REGARDING FEDERALLY SPONSORED RESEARCH OR DEVELOPMENT.

Not Applicable

### INCORPORATION-BY-REFERENCE OF MATERIAL SUBMITTED ON A COMPACT DISC.

Not Applicable

### BACKGROUND OF THE INVENTION

(1) Field of the Invention

The invention concerns cosmetic hair treatment agents based on a surfactant mixture in combination with specific hair care substances, as well as a method for simultaneous cleaning and care of the hair using said means.

Human hair is treated at present with a wide variety of cosmetic preparations that are intended to make the hair shiny and easy to comb. These various processing steps themselves, however, for example bleaching, dyeing, toning, shaping, but also the action of heat from blow-drying or mechanical stresses such as frequent combing and brushing, can result in an undesired adverse effect on the hair structure. The adverse effect on the hair structure is expressed, for example, as poor wet and dry combability, increased electrostatic charging, increased brittleness, a decrease in the maximum fracture force and strain at fracture of the hair, splitting, and a generally degraded external appearance of the hair. To counteract these disadvantages, a post-treatment with conditioning agents is absolutely necessary; this in turn makes the user dissatisfied with the enormous time expenditure for a complete hair treatment.

This problem has been partly alleviated in recent years with the development of so-called two-in-one shampoos, since these shampoos encompass cleaning and conditioning agents and thus make possible two treatment steps in one. The disadvantage of these two-in-one shampoos is, however, that the hair often becomes “over-treated”0 as a result of regular treatment with the two-in-one formulations. The volume and body of the hair, in particular, suffer as a result. In addition, subsequent treatments such as coloring or permanent waving can be adversely affected.

### BRIEF SUMMARY OF THE INVENTION

The goal was therefore to develop a novel hair treatment agent with which simultaneous hair cleaning and care is possible without thereby causing the hair to lose attractiveness because of insufficient volume.

It has now been found, entirely surprisingly, that the disadvantages of the existing art can be significantly alleviated by way of a combination of a specific surfactant mixture with selected hair care substances.

The subject matter of the invention is therefore cosmetic hair treatment agents containing
a) a surfactant mixture that is made up of
   A) at least one anionic surfactant;
   B) at least one amphoteric surfactant; and
   C) at least one further surfactant selected from the group of the acyl glutamates, the aminoxides, and the alkyl polyglucosides, as well as
b) at least two further hair care substances selected from the group of the
   fatty alcohols;
   cationic surfactants;
   cationic polymers;
   cationically derivatized protein hydrolysates;
   water-insoluble volatile silicones and/or the water-soluble volatile silicones;
   vitamins and/or provitamins and/or their physiologically acceptable derivatives.

### BRIEF DESCRIPTION OF THE SEVERAL VIEWS OF THE DRAWING(S)

Not Applicable

### DETAILED DESCRIPTION OF THE INVENTION

Preferred anionic surfactants are selected, according to the present invention, from the group of the ethoxylated alkyl sulfate surfactants, the alkyl sulfates, or the salts of the ethercarboxylic acids, and from mixtures of these substances. These anionic surfactants are preferably salts of ethercarboxylic acids of the formula R—O—(CH₂—CH₂O)ₓ—CH₂—COOH, in which R is a linear alkyl group having 8 to 30 carbon atoms and X=0 or is 1 to 16, or alkyl sulfates or alkyl polyglycol ether sulfates of the formula R—(O—CH₂—CH₂)ₓ—OSO₃H, in which R is a preferably linear alkyl group having 8 to 30 carbon atoms and X=0 or is 1 to 12.

Amphoteric surfactants preferred according to the present invention are those from the group of the alkyl betaines, amidoalkyl betaines, amphoacetates, or amphodiacetates, and from mixtures of these substances. The amphoteric surfactant classes are preferably compounds that contain, in addition to a C₈-C₂₄ alkyl or acyl group in the molecule, at least one free amino group and at least one —COOH or —SO₃H group, and are capable of forming internal salts. Examples of suitable amphoteric surfactants are N-alkyl glycines, N-alkyl propionic acids, N-alkylaminobutyric acids, N-alkyliminodipropionic acids, N-hydroxyethyl-N-alkyl amidopropyl glycines, N-alkyl taurines, N-alkyl sarcosines, 2-alkylaminopropionic acids, and alkylaminoacetic acids, each having 8 to 24 carbon atoms in the alkyl group. Particularly preferred amphoteric surfactants are N-cocalkylaminopropionate, cocacylaminoethylaminopropionate, and C₁₂-C₁₈ acyl sarcosine.

In a preferred embodiment of the invention an aminoxide is used as the third surfactant component.

“Aminoxides” are understood, according to the present invention, to be substances of the general formula R¹R²R³N—O, in which the substituents R¹, R², R³ denote, independently of one another, alkyl groups having 1 to 22 carbon atoms or alkylamidoalkyl groups having 8 to 25 carbon atoms, with the stipulation that at least one of these substituents contains at least 8 carbon atoms. An aminoxide preferred according to the present invention is the commercial product Aminoxid WS35, an N,N-dimethyl-N(C8-18-cocacylamidopropyl)amine-N-oxide having the INCl designation cocamidopropylamine oxide.

In a further preferred embodiment of the invention, a mixture of acyl glutamates and aminoxides, or a mixture of acyl glutamates and alkyl polyglucosides, is used as the third surfactant component.

Acyl glutamates constitute known anionic surfactants that conform to formula (I): 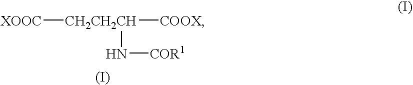
in which R¹CO denotes a linear or branched acyl radical having 6 to 22 carbon atoms and 0 and/or 1, 2, or 3 double bonds, and X denotes hydrogen, an alkali or alkaline-earth metal, ammonium, alkylammonium, alkanolammonium, or glucammonium. It is manufactured, for example, by Schotten-Baumann acylation of glutamic acid with fatty acids, or fatty acid esters or chlorides. Commercial products are obtainable, for example, from Hoechst A G, Frankfurt (Germany) or Ajinomoto Co. Inc., Tokyo (Japan). An overview of the manufacture and properties of the acyl glutamates by M. Takehara et al. may be found in J.Am.Oil Chem.Soc. 49, 143 (1982). Typical examples of suitable acyl glutamates that are appropriate for purposes of the invention are anionic surfactants that derive from fatty acids having 6 to 22, preferably 12 to 18 carbon atoms, for example C_{12/14} or C_{12/18} coconut fatty acid, lauric acid, myristic acid, palmitic acid, and/or stearic acid. Sodium N-cocoyl- and sodium N-stearoyl-L-glutamate are particularly preferred.

Alkyl polyglycosides correspond to the general formula RO-(Z)ₓ, where R denotes alkyl, Z sugar, and x the number of sugar units. The alkyl polyglycosides usable according to the present invention may contain only one specific alkyl radical R. Usually, however, these compounds are manufactured from natural fats and oils or mineral oils. In this case what is present as alkyl radicals R are mixtures corresponding to the initial compounds or corresponding to the particular processing of those compounds.

Particularly preferred are those alkyl polyglycosides in which R comprises
substantially C₈ and C₁₀ alkyl groups;
substantially C₁₂ and C₁₄ alkyl groups;
substantially C₈ to C₁₆ alkyl groups; or
substantially C₁₂ to C₁₆ alkyl groups; or
substantially C₁₆ to C₁₈ alkyl groups.

Any mono- or oligosaccharides can be used as the sugar module Z. Sugars having 5 or 6 carbon atoms, as well as the corresponding oligosaccharides, are usually used. Such sugars are, for example, glucose, fructose, galactose, arabinose, ribose, xylose, lyxose, allose, altrose, mannose, gulose, idose, talose, and sucrose. Preferred sugar modules are glucose, fructose, galactose, arabinose, and sucrose; glucose is particularly preferred.

The alkyl polyglycosides usable according to the present invention contain on average 1.1 to 5 sugar units. Alkyl polyglycosides having values of x from 1.1 to 2.0 are preferred. Alkyl polyglycosides in which x equals 1.1 to 1.8 are very particularly preferred.

The alkoxylated homologs of the aforesaid alkyl polyglycosides can also be used according to the present invention. These homologs can contain, on average, up to 10 ethylene oxide and/or propylene oxide units per alkyl glycoside unit.

A ratio of the surfactant components (A):(B):(C) of (3-6):1:(1-2) is preferred according to the present invention.

The hair care substances b) can be selected, according to the present invention, both from one and from several of the groups listed. It is preferred according to the present invention, however, if at least two representatives of at least two groups are present in the agents.

The following can be used as fatty alcohols according to the present invention: saturated, singly or multiply unsaturated, branched or unbranched fatty alcohols having C₆ to C₃₀, preferably C₁₀ to C₂₂, and very particularly preferably C₁₂ to C₂₂ carbon atoms. Usable in the context of the invention are, for example, decanol, octanol, octenol, dodecenol, decenol, octadienol, dodecadienol, decadienol, oleyl alcohol, erucic alcohol, ricinol alcohol, stearyl alcohol, isostearyl alcohol, cetyl alcohol, lauryl alcohol, myristyl alcohol, arachidyl alcohol, capryl alcohol, capric alcohol, linoleyl alcohol, linolenyl alcohol, and behenyl alcohol, as well as Guerbet alcohols thereof, this listing being intended to be exemplary and not limiting in nature. The fatty alcohols preferably derive, however, from natural fatty acids; it is usually possible to proceed from an extraction from the esters of the fatty acids by reduction. Also usable according to the present invention are those fatty alcohol cuts that are generated by the reduction of naturally occurring triglycerides such as beef tallow, palm oil, peanut oil, colza oil, cottonseed oil, soybean oil, sunflower oil, and linseed oil, or from fatty acid esters resulting from their transesterification products with corresponding alcohols. Such substances are, for example, available commercially under the designations Stenol®, e.g., Stenol® 1618, or Lanette®, e.g., Lanette®, or Lorol®, e.g., Lorol® C8, Lorol® C14, Lorol® C18, Lorol® C8-18, HD-Ocenol®, Crodacol®, e.g., Crodacol® CS, Novol®, Eutanol® G, Guerbitol® 16, Guerbitol® 18, Guerbitol® 20, Isofol® 12, Isofol® 16 Isofol® 24, Isofol® 36, Isocarb® 12, Isocarb® 16, or Isocarb® 24. It is of course also possible according to the present invention to use wool-wax alcohols such as those available commercially under the designations Corona®, White Swan®, Coronet®, or Fluilan®.

Fatty alcohols that are very particularly preferred according to the present invention are cetyl alcohol, stearyl alcohol, or mixtures of these components.

The fatty alcohols are used in quantities of 0.1-20 wt % relative to the entire preparation, preferably in quantities of 0.1-10 wt %.

“Cationic surfactants” for purposes of the invention are preferably to be understood as cationic surfactants of the following types: quaternary ammonium compounds, esterquats, and amide amines. Preferred quaternary ammonium compounds are ammonium halides, in particular chlorides and bromides, such as alkyl trimethylammonium chlorides, dialkyl dimethylammonium chlorides, and trialkyl methylammonium chlorides, e.g., cetyl trimethylammonium chloride, stearyl trimethylammonium chloride, distearyl dimethylammonium chloride, lauryl dimethylammonium chloride, lauryl dimethylbenzylammonium chloride, and tricetyl methylammonium chloride, as well as the imidazolium compounds known under the INCl designations quaternium-27 and quaternium-83. The long alkyl chains of the aforementioned surfactants preferably have 10 to 18 carbon atoms.

Esterquats are known substances that contain both at least one ester function and at least one quaternary ammonium group as structural elements. Preferred esterquats are quaternized ester salts of fatty acids with triethanolamine, quaternized ester salts of fatty acids with diethanolalkylamines, and quaternized ester salts of fatty acids with 1,2-dihydroxypropyldialkylamines. Such products are marketed, for example, under the trademarks Stepantex®, Dehyquart®, and Armocare®. The products Armocare® VGH-70, an N,N-bis(2-palmitoyloxyethyl)dimethylammonium chloride, as well as Dehyquart® F-75, Dehyquart® C-4046, Dehyquart® L80, and Dehyquart® AU-35, are examples of such esterquats.

The alkylamide amines are usually produced by amidation of natural or synthetic fatty acids and fatty acid cuts with dialkylaminoamines. A compound from this substance group that is particularly suitable according to the present invention is represented by the stearamidopropyl dimethylamine available commercially under the designation Tegoamid® S 18.

The cationic surfactants are contained in the agents used according to the present invention preferably in quantities of 0.05 to 10 wt % relative to the entire agent. Quantities from 0.1 to 5 wt % are particularly preferred.

“Cationic polymers” are understood for purposes of the invention to be, for example, cationic cellulose derivatives such as, for example, a quaternized hydroxyethylcellulose that is available from Amerchol under the designation Polymer JR® 400, cationic starch, copolymers of diallylammonium salts and acrylamides, quaternized vinylpyrrolidone/vinylimidazole polymers such as Luviquat® (BASF), condensation products of polyglycols and amines, quaternized collagen polypeptides such as lauryldimonium hydroxypropyl hydrolyzed collagen (Lamequat® L, Grucnau), quaternized wheat polypeptides, polyethylene imine, cationic silicone polymers such as amidodimethicone, copolymers of adipic acid and dimethylaminohydroxypropyldiethylenetriamine (Cartaretine®, Sandoz), copolymers of acrylamide with dimethyldiallylammonium chloride (Merquat® 550, Chemviron), homopolymers of dimethyldiallylammonium chloride (Merquat® 100), polyaminopolyamides such as those described, for example, in FR-A 2 252 840 and their cross-linked water-soluble polymers, cationic chitin derivatives such as quaternized chitosan, optionally distributed in microcrystalline fashion, condensation products of dihalogen alkylene, for example dibromobutane with bisdialkylamines, for example bis(dimethylamino)-1,3-propane, cationic guar gum such as Jaguar® CBS, Jaguar® C-17, Jaguar® C-16 of the Celanese company, quaternized ammonium salt polymers, for example Mirapol® A-15, Mirapol® AD-1, Mirapol® AZ-1 of the Miranol company.

Particularly preferred cationic polymers are cationic guar derivatives, cationic cellulose derivatives, homopolymers of dimethyldiallylammonium chloride, and copolymers of dimethyldiallylammonium chloride with acrylamide.

Polyquaternium-10 is a very particularly preferred cationic polymer for purposes of the invention.

The cationic polymers are used in the compositions according to the present invention usually in a quantity of 0.001 to 20 wt %, preferably in a quantity of 0.01 to 5 wt %, and in particular in a quantity of 0.1 to 2 wt %, in each case relative to the total weight of the agent.

The protein hydrolysates for purposes of the invention are usually protein hydrolysates from animals, for example from collagen, milk, or keratin, from plants, for example from wheat, corn, rice, potatoes, soybeans, or almonds, from marine life forms, for example fish collagen or algae, or from biotechnologically obtained protein hydrolysates. The protein hydrolysates on which the cationic derivatives according to the present invention are based can be obtained from the corresponding proteins by way of a chemical, in particular alkaline or acid, hydrolysis, by an enzymatic hydrolysis, and/or by a combination of the two types of hydrolysis. The hydrolysis of proteins generally yields a protein hydrolysate having a molecular weight distribution from approximately 100 daltons to several thousand daltons. Those cationic protein hydrolysates whose underlying protein fraction has a molecular weight of 100 to 25,000 daltons, preferably 250 to 5000 daltons, are preferred. Quaternized amino acids and mixtures thereof are also to be understood as cationic protein hydrolysates. The quaternization of protein hydrolysates or of amino acids is often carried out by means of quaternary ammonium salts such as N,N-dimethyl-N-(n-alkyl)-N-(2-hydroxy-3-chloro-n-propyl)ammonium halides. The cationic protein hydrolysates can moreover be even further derivatized.

Typical examples of the cationic protein hydrolysates and derivatives according to the present invention that may be mentioned are the products cited under their INCl designations in the “International Cosmetic Ingredient Dictionary and Handbook,” (seventh edition 1997, The Cosmetic, Toiletry, and Fragrance Association, 1101 17th St., NW, Suite 300, Washington, D.C. 20036-4702), and available commercially: cocodimonium hydroxypropyl hydrolyzed collagen, cocodimonium hydroxypropyl hydrolyzed casein, cocodimonium hydroxypropyl hydrolyzed collagen, cocodimonium hydroxypropyl hydrolyzed hair keratin, cocodimonium hydroxypropyl hydrolyzed keratin, cocodimonium hydroxypropyl hydrolyzed rice protein, cocodimonium hydroxypropyl hydrolyzed silk, cocodimonium hydroxypropyl hydrolyzed soy protein, cocodimonium hydroxypropyl hydrolyzed wheat protein, cocodimonium hydroxypropyl silk amino acids, hydroxypropyl arginine lauryl/myristyl ether HCl, hydroxypropyltrimonium hydrolyzed gelatin, hydroxypropyltrimonium hydrolyzed casein, hydroxypropyltrimonium hydrolyzed collagen, hydroxypropyltrimonium hydrolyzed conchiolin protein, hydroxypropyltrimonium hydrolyzed keratin, hydroxypropyltrimonium hydrolyzed rice bran protein, hydroxypropyltrimonium hydrolyzed silk, hydroxypropyltrimonium hydrolyzed soy protein, hydroxypropyltrimonium hydrolyzed vegetable protein, hydroxypropyl hydrolyzed wheat protein, hydroxypropyltrimonium hydrolyzed wheat protein/siloxysilicate, laurdimonium hydroxypropyl hydrolyzed soy protein, laurdimonium hydroxypropyl hydrolyzed wheat protein, laurdimonium hydroxypropyl hydrolyzed wheat protein/siloxysilicate, laurdimonium hydroxypropyl hydrolyzed casein, laurdimonium hydroxypropyl hydrolyzed collagen, laurdimonium hydroxypropyl hydrolyzed keratin, laurdimonium hydroxypropyl hydrolyzed silk, laurdimonium hydroxypropyl hydrolyzed soy protein, steardimonium hydroxypropyl hydrolyzed casein, steardimonium hydroxypropyl hydrolyzed collagen, steardimonium hydroxypropyl hydrolyzed keratin, steardimonium hydroxypropyl hydrolyzed rice protein, steardimonium hydroxypropyl hydrolyzed silk, steardimonium hydroxypropyl hydrolyzed soy protein, steardimonium hydroxypropyl hydrolyzed vegetable protein, steardimonium hydroxypropyl hydrolyzed wheat protein, steardimonium hydroxyethyl hydrolyzed collagen, quaternium-76 hydrolyzed collagen, quaternium-79 hydrolyzed collagen, quaternium-79 hydrolyzed keratin, quaternium-79 hydrolyzed milk protein, quaternium-79 hydrolyzed silk, quaternium-79 hydrolyzed soy protein, quaternium-79 hydrolyzed wheat protein.

The cationic protein hydrolysates and derivatives of keratin, collagen, elastin, soy, milk, wheat, silk, and almonds are very particularly preferred; hydroxypropyl hydrolyzed wheat protein, as available commercially under the trade name Gluadin WQ from the Cognis company, is particularly preferred.

The protein hydrolysates and their derivatives are contained in the agents used according to the present invention in quantities of 0.01 to 10 wt % relative to the entire agent. Quantities of 0.1 to 5 wt %, in particular 0.1 to 3 wt %, are very particularly preferred.

“Water-insoluble silicones” are understood for purposes of the invention to be those silicones that are soluble to a maximum of 0.5% in water at 20° C.

“Volatile silicones” are understood for purposes of the invention to be those that evaporate completely and leave no residue on the substrate. Those silicones of which a 10 g sample on a Petri dish is 5-90% evaporated at 60° C. within 120 minutes, are preferred. Those silicones that are 10-50% evaporated under those conditions are particularly preferred.

Silicone compounds that are preferred according to the present invention are those according to formula (II): 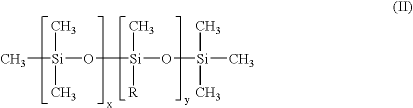
in which x denotes a number from 1 to 10, y denotes a number from 1 to 10, and R denotes an alkyl radical having 2 to 10 carbon atoms.

Those silicone compounds according to formula (II) in which the numbers x and y denote the number 1 are particularly preferred.

Particularly preferred silicone components for purposes of the invention are the compounds hexyl methicone and caprylyl methicone, for example such as those obtainable from the Clariant company under the trade names SilCare 41M10 and SilCare 41M15.

The silicone components are used in the agents according to the present invention usually in a quantity from 0.001 to 20 wt %, preferably in a quantity from 0.1 to 10 wt %, and in particular in a quantity from 0.5 to 2 wt %, in each case relative to the total weight of the agent.

Vitamins, provitamins, and vitamin precursors according to the present invention are usually selected from the A, B, C, E, F, and H groups.

The group of substances referred to as vitamin A includes retinol (vitamin A₁) as well as 3,4-didehydroretinol (vitamin A₂). β-carotene is the provitamin of retinol. Suitable vitamin A components according to the present invention are, for example, vitamin A acid and its esters, vitamin A aldehyde, and vitamin A alcohol, as well as its esters such as the palmitate and the acetate. The preparations used according to the present invention preferably contain the vitamin A component in quantities from 0.05 to 1 wt % relative to the entire preparation.

The vitamin B group or the vitamin B complex includes, among others:

Vitamin B₁ (thiamine).

Vitamin B₂ (riboflavin).

Vitamin B₃. The compounds nicotinic acid and nicotinic acid amide (niacinamide) are often listed under this designation. Nicotinic acid amide, which is contained in the agents used according to the present invention preferably in quantities from 0.05 to 1 wt % relative to the entire agent, is preferred according to the present invention.

Vitamin B₅ (pantothenic acid, panthenol, and pantolactone). In the context of this group, panthenol and/or pantolactone are preferably used. Derivatives of panthenol that are usable according to the present invention are, in particular, the esters and ethers of panthenol as well as cationically derivatized panthenols. Individual representatives are, for example, panthenol triacetate, panthenol monoethyl ester and its monoacetate, and the cationic panthenol derivatives disclosed in WO 92/13829. The aforesaid compounds of the vitamin B₅ type are contained in the agents used according to the present invention preferably in quantities of 0.05 to 10 wt % relative to the entire agent. Quantities of 0.1 to 5 wt % are particularly preferred.

Vitamin B₆ (pyridoxine, as well as pyridoxamine and pyridoxal).

Vitamin C (ascorbic acid). Vitamin C is utilized in the agents used according to the present invention preferably in quantities from 0.1 to 3 wt % relative to the entire agent. Utilization in the form of the palmitic acid ester, the glucosides, or phosphates can be preferred. Utilization in combination with tocopherols can likewise be preferred.

Vitamin E (tocopherols, in particular α-tocopherol). Tocopherol and its derivatives, which include in particular the esters such as the acetate, nicotinate, phosphate, and succinate, are contained in the agents used according to the present invention preferably in quantities from 0.05 to 1 wt % relative to the entire agent.

Vitamin F. The term “vitamin F” is usually understood to mean essential fatty acids, in particular linoleic acid, linolenic acid, and arachidonic acid.

Vitamin H. What is referred to as “vitamin H” is the compound (3aS,4S,6aR)-2-oxohexahydrothienol[3,4-d]-imidazol-4-valeric acid, for which the trivial name “biotin” has nevertheless now become established. Biotin is contained in the agents used according to the present invention preferably in quantities of 0.0001 to 1.0 wt %, in particular in quantities of 0.001 to 0.01 wt %.

The preparations used according to the present invention preferably contain vitamins, provitamins, and vitamin precursors from groups A, B, E, and H. Multiple vitamins and vitamin precursors can, of course, also be present.

Pantolactone, pyridoxine and its derivatives, and nicotinic acid amide and biotin, but in particular panthenol and its physiologically acceptable derivatives, are particularly preferred.

It may be preferred according to the present invention to utilize additional further surfactants from the group of the anionic, cationic, zwitterionic, amphoteric, or nonionic surfactants.

Suitable as further anionic surfactants in preparations according to the present invention are all anionic surface-active substances suitable for use on the human body. These are characterized by an anionic group that creates water-solubility, such as a carboxylate, sulfate, sulfonate, or phosphate group, and a lipophilic alkyl group having approximately 8 to 30 carbon atoms. Glycol ether or polyglycol ether groups, ester, ether, and amide groups, and hydroxyl groups can additionally be contained in the molecule. Examples of suitable anionic surfactants are, in each case in the form of the sodium, potassium, and ammonium salts, and the mono-, di-, and trialkanolammonium salts having 2 to 4 carbon atoms in the alkanol group:
linear and branched fatty acids having 8 to 30 carbon atoms (soaps);
acyl sarcosides having 8 to 24 carbon atoms in the acyl group;
acyl taurides having 8 to 24 carbon atoms in the acyl group;
acyl isethionates having 8 to 24 carbon atoms in the acyl group;
sulfosuccinic acid mono- and dialkyl esters having 8 to 24 carbon atoms in the alkyl group, and sulfosuccinic acid monoalkylpolyoxyethyl esters having 8 to 24 carbon atoms in the alkyl group and 1 to 6 oxyethyl groups;
linear alkanesulfonates having 8 to 24 carbon atoms;
linear alpha-olefinsulfonates having 8 to 24 carbon atoms;
alpha-sulfo fatty acid methyl esters of fatty acids having 8 to 30 carbon atoms;
mixtures of surface-active hydroxysulfonates according to DE-A-37 25 030;
sulfated hydroxyalkylpolyethylene and/or hydroxyalkylenepropylene glycol ethers according to DE-A-37 23 354;
sulfonates of unsaturated fatty acids having 8 to 24 carbon atoms and 1 to 6 double bonds, according to DE-A-39 26 344;
esters of tartaric acid and citric acid with alcohols, representing addition products of approximately 2-15 molecules of ethylene oxide and/or propylene oxide with fatty alcohols having 8 to 22 carbon atoms;
alkyl and/or alkylene ether phosphates of formula (III): 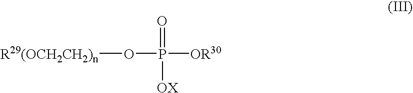
   in which R²⁹ preferably denotes an aliphatic hydrocarbon radical having 8 to 30 carbon atoms, R³⁰ denotes hydrogen, a (CH₂CH₂O)ₙR²⁹ radical, or X, n denotes numbers from 1 to 10, and X denotes hydrogen, an alkaline or alkaline-earth metal, or NR³¹R³²R³³R³⁴, where R³¹ to R³⁴, independently of one another, denote a C₁ to C₄ hydrocarbon radical;
sulfated fatty acid alkylene glycol esters of formula (IV):
   R³⁵CO(AlkO)ₙSO₃M (IV)
   in which R³⁵CO denotes a linear or branched, aliphatic, saturated and/or unsaturated acyl radical having 6 to 22 carbon atoms, Alk denotes CH₂CH₂, CHCH₃CH₂, and/or CH₂CHCH₃, n denotes numbers from 0.5 to 5, and M denotes a cation such as those described in DE-OS 197 36 906.5;
monoglyceride sulfates and monoglyceride ether sulfates of formula (V), such as those that have been described in EP-B1 0 561 825, EP-B1 0 561 999, DE-A1 42 04 700, or by A. K. Biswas et al. in J. Am. Oil Chem. Soc. 37, 171 (1960) and F. U. Ahmed in J. Am. Oil Chem. Soc. 67, 8 (1990): 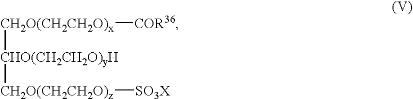
   in which R³⁶CO denotes a linear or branched acyl radical having 6 to 22 carbon atoms, x, y, and z in total denote 0 or numbers from 1 to 30, preferably 2 to 10, and X denotes an alkali or alkaline-earth metal. Typical examples of monoglyceride (ether) sulfates suitable for purposes of the invention are the reaction products of lauric acid monoglyceride, coconut fatty acid monoglyceride, palmitic acid monoglyceride, stearic acid monoglyceride, oleic acid monoglyceride, and tallow fatty acid monoglyceride, as well as their ethylene oxide adducts with sulfur trioxide or chlorosulfonic acid in the form of their sodium salts. Monoglyceride sulfates of formula (V) in which R³⁶CO denotes a linear acyl radical having 8 to 18 carbon atoms are preferably used.

Preferred further anionic surfactants are sulfosuccinic acid mono- and dialkyl esters having 8 to 18 carbon atoms in the alkyl group, and sulfosuccinic acid monoalkylpolyoxyethyl esters having 8 to 18 carbon atoms in the alkyl group and 1 to 6 oxyethyl groups.

The term “zwitterionic surfactants” refers to those surface-active compounds that carry in the molecule at least one quaternary ammonium group and at least one —COO⁽⁻⁾ or —SO₃⁽⁻⁾ group. Particularly suitable zwitterionic surfactants are the so-called betaines, such as N-alkyl-N,N-dimethylammonium glycinate, for example cocalkyldimethylammonium glycinate, N-acylaminopropyl-N,N-dimethylammonium glycinate, for example cocacylaminopropyidimethylammonium glycinate, and 2-alkyl-3-carboxymethyl-3-hydroxyethyl imidazoline, in each case having 8 to 18 carbon atoms in the alkyl or acyl group, as well as cocacylaminoethylhydroxyethyl-carboxymethyl glycinate. A preferred zwitterionic surfactant is the fatty acid amide derivative known under the INCl designation cocamidopropyl betaine.

Further nonionic surfactants contain as the hydrophilic group, for example, a polyol group, a polyalkylene glycol ether group, of a combination of a polyol and polyglycol ether group. Such compounds are, for example:
addition products of 2 to 50 mol ethylene oxide and/or 0 to 5 mol propylene oxide with linear and branched fatty alcohols having 8 to 30 carbon atoms, with fatty acids having 8 to 30 carbon atoms, and with alkyl phenols having 8 to 15 carbon atoms in the alkyl group;
addition products, having a methyl or C₂ to C₆ alkyl terminating group, of 2 to 50 mol ethylene oxide and/or 0 to 5 mol propylene oxide with linear and branched fatty alcohols having 8 to 30 carbon atoms, with fatty acids having 8 to 30 carbon atoms, and with alkyl phenols having 8 to 15 carbon atoms in the alkyl group, for example the types obtainable under the marketing designations Dehydol® LS, Dehydol® LT (Cognis);
C₁₂-C₃₀ fatty acid mono- and diesters of addition products of 1 to 30 mole ethylene oxide with glycerol;
addition products of 5 to 60 mol ethylene oxide with castor oil and hardened castor oil;
polyol fatty acid esters, for example the commercial product Hydagene HSP (Cognis), or Sovermol grades (Cognis);
alkoxylated triglycerides;
alkoxylated fatty acid alkyl esters of formula (VI):
   R³⁷CO—(OCH₂CHR³⁸)_{w}OR³⁹ (VI),
   in which R³⁷CO denotes a linear or branched, saturated and/or unsaturated acyl radical having 6 to 22 carbon atoms, R³⁸ denotes hydrogen or methyl, R³⁹ denotes linear or branched alkyl radicals having 1 to 4 carbon atoms, and w denotes numbers from 1 to 20;
hydroxy mixed ethers, such as those described e.g., in DE-OS 197 38 866;
sorbitan fatty acid esters and addition products of ethylene oxide with sorbitan fatty acid esters, for example the polysorbates;
sugar fatty acid esters and addition products of ethylene oxide with sugar fatty acid esters;
addition products of ethylene oxide with fatty acid alkanolamides and fatty amines;
fatty acid N-alkyl glucamides.

The alkylene oxide addition products with saturated linear fatty alcohols and fatty acids, having respectively 2 to 30 mol ethylene oxide per mol fatty alcohol or fatty acid, have proven to be preferred further nonionic surfactants. Preparations having outstanding properties are also obtained if they contain, as nonionic surfactants, fatty acid esters of ethoxylated glycerol.

These compounds are characterized by the following parameters: The alkyl radical R contains 6 to 22 carbon atoms and can be both linear and branched. Primary linear aliphatic radicals, methyl-branched in the 2-position, are preferred. Such alkyl radicals are, for example, 1-octyl, 1-decyl, 1-lauryl, 1-myristyl, 1-cetyl, and 1-stearyl. 1-octyl, 1-decyl, 1-lauryl, and 1-myristyl are particularly preferred. When so-called “oxo alcohols” are used as the initial materials, compounds having an odd number of carbon atoms in the alkyl chain predominate.

The compounds having alkyl groups used as surfactants can in each case be uniform substances. It is generally preferred, however, to begin with natural vegetable or animal raw materials when producing these substances, so that substance mixtures having different alkyl chain lengths, dependent on the particular material, are obtained.

In the surfactants that represent addition products of ethylene and/or propylene oxide with fatty alcohols, or derivatives of such addition products, both products having a “normal” homolog distribution and those having a restricted homolog distribution can be used. A “normal” homolog distribution is understood to mean mixtures of homologs that are obtained when reacting fatty alcohol and alkylene oxide using alkali metals, alkali metal hydroxides, or alkali metal alcoholates as catalysts. Restricted homolog distributions, on the other hand, are obtained when, for example, hydrotalcites, alkaline-earth metal salts or ethercarboxylic acids, alkaline-earth metal oxides, hydroxides, or alcoholates are used as catalysts. The use of products having a restricted homolog distribution can be preferred.

The surfactants are used in quantities of 0.1 to 45 wt %, preferably 1 to 30 wt %, and very particularly preferably 1 to 15 wt %, relative to the entire agent.

Also preferred according to the present invention are cationic surfactants of the following types: quaternary ammonium compounds, esterquats, and amide amines. Preferred quaternary ammonium compounds are ammonium halides, in particular chlorides and bromides, such as alkyl trimethylammonium chlorides, dialkyl dimethylammonium chlorides, and trialkyl methylammonium chlorides, e.g., cetyl trimethylammonium chloride, stearyl trimethylammonium chloride, distearyl dimethylammonium chloride, lauryl dimethylammonium chloride, lauryl dimethylbenzylammonium chloride, and tricetyl methylammonium chloride, as well as the imidazolium compounds known under the INCl designations quaternium-27 and quaternium-83. The long alkyl chains of the aforementioned surfactants preferably have 10 to 18 carbon atoms.

Esterquats are known substances that contain both at least one ester function and at least one quaternary ammonium group as structural elements. Preferred esterquats are quaternized ester salts of fatty acids with triethanolamine, quaternized ester salts of fatty acids with diethanolalkylamines, and quaternized ester salts of fatty acids with 1,2-dihydroxypropyldialkylamines. Such products are marketed, for example, under the trademarks Stepantex®, Dehyquart®, and Armocare®. The products Armocare® VGH-70, an N,N-bis(2-palmitoyloxyethyl)dimethylammonium chloride, as well as Dehyquart® F-75, Dehyquart® C-4046, Dehyquart® L80, and Dehyquart® AU-35, are examples of such esterquats.

The alkylamide amines are usually produced by amidation of natural or synthetic fatty acids and fatty acid cuts with dialkylaminoamines. A compound from this substance group that is particularly suitable according to the present invention is represented by the stearamidopropyl dimethylamine available commercially under the designation Tegoamid® S 18.

The cationic surfactants are contained in the agents used according to the present invention preferably in quantities of 0.05 to 10 wt % relative to the entire agent. Quantities from 0.1 to 5 wt % are particularly preferred.

In a further preferred embodiment, the effect of the agent according to the present invention can be enhanced by emulsifiers. Such emulsifiers are, for example:
addition products of 4 to 30 mol ethylene oxide and/or 0 to 5 mol propylene oxide with linear fatty alcohols having 8 to 22 carbon atoms, with fatty acids having 12 to 22 carbon atoms, and with alkyl phenols having 8 to 15 carbon atoms in the alkyl group;
C₁₂-C₂₂ fatty acid mono- and diesters of addition products of 1 to 30 mol ethylene oxide with polyols having 3 to 6 carbon atoms, in particular with glycerol;
ethylene oxide and polyglycerol addition products with methyl glucoside fatty acid esters, fatty acid alkanolamides, and fatty acid glucamides;
C₈-C₂₂ alkyl mono- and oligoglycosides and their ethyoxylated analogs, degrees of oligomerization of 1.1 to 5, in particular 1.2 to 2.0, and glucose as the sugar component, being preferred;
mixtures of alkyl (oligo)glucosides and fatty alcohols, for example the commercially available product Montanov® 68;
addition products of 5 to 60 mol ethylene oxide with castor oil and hardened castor oil;
partial esters of polyols having 3 to 6 carbon atoms with saturated fatty acids having 8 to 22 carbon atoms;
Sterols. “Sterols” are understood to mean a group of steroids that carry a hydroxyl group on the third carbon atom of the steroid structure and are isolated both from animal tissue (zoosterols) and from vegetable fats (phytosterols). Examples of zoosterols are cholesterol and lanosterol. Examples of suitable phytosterols are ergosterol, stigmasterol, and sitosterol. Sterols are also isolated from fungi and yeasts (the so-called mycosterols).
Phospholipids. These are understood to mean principally the glucose phospholipids, which are obtained e.g., as lecithins or phosphatidylcholines from for example, egg yolk or plant seeds (e.g., soybeans).
Fatty acid esters of sugar and sugar alcohols, such as sorbitol, polyglycerols and polyglycerol derivatives such as polyglycerol poly-12-hydroxystearate (commercial product Dehymuls® PGPH).
Linear and branched fatty acids having 8 to 30 carbon atoms, and their Na, K, ammonium, Ca, Mg, and Zn salts.

The agents according to the present invention contain the emulsifiers preferably in quantities from 0.1 to 25 wt %, in particular 0.5 to 15 wt %, relative to the entire agent.

The compositions according to the present invention can preferably contain at least one nonionogenic emulsifier having an HLB value from 8 to 18, according to the definitions set forth in the Römpp-Lexikon Chemie [Römpp chemical dictionary] (J. Falbe, M. Regitz, eds.), 10th edition, Georg Thieme Verlag Stuttgart, New York (1997), page 1764. Nonionogenic emulsifiers having an HLB value from 10 to 15 may be particularly preferred according to the present invention.

Among the aforesaid types of emulsifiers, those emulsifiers that contain no ethylene oxide and/or propylene oxide in the molecule may be very particularly preferred.

In a further preferred embodiment, the effect of the agent according to the present invention can be enhanced by oily substances. Such oily substances and/or fatty substances are to be understood, for example, as fatty acids, natural and synthetic waxes that can be present both in solid form and in liquid form in aqueous dispersion, and natural and synthetic cosmetic oil components.

The fatty acids that can be used are linear and/or branched, saturated and/or unsaturated fatty acids having 6 to 30 carbon atoms. Fatty acids having 10 to 22 carbon atoms are preferred. Among those that might be mentioned are, for example, the isostearic acids, such as the commercial products Emersol® 871 and Emersol® 875, and isopalmitic acids such as the commercial product Edenor® IP 95, as well as all other fatty acids marketed under the Edenor® commercial designations (Cognis). Further typical examples of such fatty acids are hexanoic acid, octanoic acid, 2-ethylhexanoic acid, decanoic acid, lauric acid, isotridecanoic acid, myristic acid, palmitic acid, palmitoleic acid, stearic acid, isostearic acid, oleic acid, elaidic acid, petroselinic acid, linoleic acid, linolenic acid, elaeostearic acid, arachidic acid, gadoleic acid, behenic acid, and erucic acid, as well as industrial mixtures thereof that occur, for example, upon high-pressure cleavage of natural fats and oils, oxidation of aldehydes from Roelen oxosynthesis, or dimerization of unsaturated fatty acids. The fatty acid cuts that are obtainable from coconut oil or palm oil are usually particularly preferred; the use of stearic acid is, as a rule, particularly preferred.

The quantity used is 0.1 to 15 wt % relative to the entire agent. In a preferred embodiment, the quantity is 0.5 to 10 wt %, quantities from 1 to 5 wt % being very particularly advantageous.

Solid paraffins or isoparaffins, carnauba waxes, beeswaxes, candelilla waxes, ozocerites, ceresin, spermaceti, sunflower wax, fruit waxes such as apple wax or citrus wax, or microcrystalline waxes made from PE or PP can be used according to the present invention as natural or synthetic waxes. Such waxes are obtainable, for example, via Kahl & Co., Trittau.

The natural and synthetic cosmetic oily substances that can enhance the effect of the active ingredient according to the present invention include, for example, vegetable oils. Examples of such oils are sunflower oil, olive oil, soybean oil, rapeseed oil, almond oil, jojoba oil, orange oil, wheat germ oil, peach-kernel oil, and the liquid components of coconut oil. Also suitable, however, are other triglyceride oils such as the liquid components of beef tallow, as well as synthetic triglyceride oils, liquid paraffin oils, isoparaffin oils, and synthetic hydrocarbons, as well as di-n-alkyl ethers having a total of between 12 and 36 carbon atoms, in particular 12 to 24 carbon atoms, for example di-n-octyl ether, di-n-decyl ether, di-n-nonyl ether, di-n-undecyl ether, di-n-dodecyl ether, n-hexyl-n-octyl ether, n-octyl-n-decyl ether, n-decyl-n-undecyl ether, n-undecyl-n-dodecyl ether, and n-hexyl-n-undecyl ether, as well as ditert-butyl ether, diisopentyl ether, di-3-ethyldecyl ether, tert-butyl-n-octyl ether, isopentyl-n-octyl ether, and 2-methylpentyl-n-octyl ether. The compounds 1,3-di-(2-ethylhexyl) cyclohexane (Cetiol® S) and di-n-octyl ether (Cetiol® OE), available as commercial products, may be preferred.

Ester oils, however, are also suitable according to the present invention. “Ester oils” are to be understood as the esters of C₆-C₃₀ fatty acids with C₂-C₃₀ fatty alcohols. The monoesters of the fatty acids with alcohols having 2 to 24 carbon atoms are preferred. Examples of fatty acid components used in the esters are hexanoic acid, octanoic acid, 2-ethylhexanoic acid, decanoic acid, lauric acid, isotridecanoic acid, myristic acid, palmitic acid, palmitoleic acid, stearic acid, isostearic acid, oleic acid, elaidic acid, petroselinic acid, linoleic acid, linolenic acid, elaeostearic acid, arachidic acid, gadoleic acid, behenic acid, and erucic acid, as well as industrial mixtures thereof that occur, for example, upon high-pressure cleavage of natural fats and oils, oxidation of aldehydes from Roelen oxosynthesis, or dimerization of unsaturated fatty acids. Examples of the fatty alcohol components in the ester oils are isopropyl alcohol, hexanol, octanol, 2-ethylhexyl alcohol, decanol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linolyl alcohol, linolenyl alcohol, elaeostearyl alcohol, arachyl alcohol, gadoleyl alcohol, behenyl alcohol, erucyl alcohol, and brassidyl alcohol, as well as industrial mixtures thereof that occur, for example, upon high-pressure hydrogenation of industrial methyl esters based on fats and oils or aldehydes from Roelen oxosynthesis, and as a monomer fraction upon dimerization of unsaturated fatty alcohols. Particularly preferred according to the present invention are isopropyl myristate (Rilanit® IPM), isononanoic acid C16-18 alkyl ester (Cetiol® SN), 2-ethylhexyl palmitate (Cegesoft® 24), stearic acid 2-ethylhexyl ester (Cetiol® 868), cetyl oleate, glycerol tricaprylate, coconut fatty alcohol caprinate/caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN), oleic acid decyl ester (Cetiol® V).

Dicarboxylic acid esters are also suitable according to the present invention. Dicarboxylic acid esters for purposes of the invention are di-n-butyl adipate, di(2-ethylhexyl)adipate, di(2-ethylhexyl)succinate, and diisotridecyl acetate, as well as diol esters such as ethylene glycol dioleate, ethylene glycol diisotridecanoate, propylene glycol di(2-ethyl hexanoate), propylene glycol diisostearate, propylene glycol dipelargonate, butanediol diisostearate, neopentyl glycol dicaprylate.

Also suitable are:
symmetrical, asymmetrical, or cyclic esters of carbon dioxide with fatty alcohols, described for example in DE-OS 197 56 454, glycerol carbonate, or dicaprylyl carbonate (Cetiol® CC);
fatty acid mono-, di-, and triesters of saturated and/or unsaturated linear and/or branched fatty acids with glycerol, for example Monomuls® 90-O18, Monomuls® 90-L12, or Cutina® Md.

Very particularly preferred according to the present invention are the oily substances from the group that is made up of ester oils of C₆-C₃₀ fatty acids with C₂-C₃₀ fatty alcohols, fatty acid glycerides from fatty acid mono-, di-, and triesters of saturated and/or unsaturated linear and/or branched C₆-C₃₀ fatty acids with glycerol, and ethoxylated fatty acid glycerides from fatty acid mono-, di-, and triesters of saturated and/or unsaturated linear and/or branched C₆-C₃₀ fatty acids with glycerol. A degree of ethoxylation from 2 to 20 is preferred.

The total quantity of oil and fat components in the agents according to the present invention is usually 2 to 25 wt % relative to the entire agent. Quantities from 4 to 25 wt % are preferred according to the present invention, and quantities from 6 to 20 wt % are particularly preferred according to the present invention.

Lastly, plant extracts (L) can be used in the agents according to the present invention.

These extracts are usually produced by extraction of the whole plants. In individual cases, however, it may also be preferred to produce the extracts exclusively from blossoms and/or leaves of the plants.

With regard to the plant extracts usable according to the present invention, reference is made in particular to the extracts listed in the table that begins on page 44 of the 3rd edition of the Leitfaden zur Inhaltsstoffdeklaration kosmetischer Mittel [Guidelines for declaring the ingredients of cosmetic agents], published by the Industrieverband Körperpflege- und Waschmittel e.V. [Federation of the Personal Hygiene and Washing Agents Industry] (IKW), Frankfurt.

Especially preferred according to the present invention are extracts from green tea, oak bark, nettle, hamamelis, hops, henna, chamomile, burdock root, horsetail, hawthorn, linden blossom, almond, aloe vera, pine needles, horse chestnut, sandalwood, juniper, coconut, mango, apricot, lemon, wheat, kiwi fruit, melon, orange, grapefruit, salvia, rosemary, birch, mallow, lady's-smock, wild thyme, yarrow, thyme, lemon balm, restharrow, coltsfoot, hibiscus, meristem, ginseng, and ginger root.

The extracts from green tea, oak bark, nettle, hamamelis, hops, chamomile, burdock root, horsetail, linden blossom, almond, aloe vera, coconut, mango, apricot, lemon, wheat, kiwi fruit, melon, orange, grapefruit, salvia, rosemary, birch, lady's-smock, wild thyme, yarrow, restharrow, meristem, ginseng, and ginger root are particularly preferred.

The extracts from green tea, almond, aloe vera, coconut, mango, apricot, lemon, wheat, kiwi fruit, and melon are very particularly suitable for the use according to the present invention.

Water, alcohol, and mixtures thereof can be used as extraction media for producing the aforesaid plant extracts. Among the preferred alcohols are the lower alcohols such as ethanol and isopropanol, but in particular the polyvalent alcohols such as ethylene glycol and propylene glycol, both alone as an extraction medium and mixed with water. Plant extracts based on water/propylene glycol at a ratio from 1:10 to 10:1 have proven particularly suitable.

The plant extracts can, according to the present invention, be used both in pure and in diluted form. If they are used in diluted form, they usually contain approx. 2 to 80 wt % active substance and, as solvent, the extraction medium or extraction medium mixture used to obtain them.

It may furthermore be preferred to use, in the agents according to the present invention, mixtures of several, in particular two, different plant extracts.

The quantity of the plant extracts utilized in the agents used according to the present invention is usually 0.01 to 50 wt % relative to the entire agent, preferably 0.1 to 30 wt %, and in particular 0.1 to 20 wt %.

Short-chain carboxylic acids (N) can additionally be used advantageously for purposes of the invention. “Short-chain carboxylic acids and their derivatives” are understood, for purposes of the invention, to mean carboxylic acids that can be saturated or unsaturated and/or straight-chain or branched or cyclic and/or aromatic and/or heterocyclic, and have a molecular weight below 750. Saturated or unsaturated straight-chain or branched carboxylic acids having a chain length of 1 to 16 carbon atoms may be preferred for purposes of the invention; those having a chain length of 1 to 12 carbon atoms in the chain are very particularly preferred.

The short-chain carboxylic acids for purposes of the invention can have one, two, three, or more carboxy groups. Carboxylic acids having multiple carboxy groups, in particular di- and tricarboxylic acids, are preferred for purposes of the invention. The carboxy groups can be present entirely or partially as an ester, acid anhydride, lactone, amide, imidic acid, lactam, lactim, dicarboximide, carbohydrazide, hydrazone, hydroxam, hydroxim, amidine, amide oxime, nitrile, phosphonic or phosphate ester. The carboxylic acids according to the present invention can, of course, be substituted along the carbon chain or the ring structure. Among the substituents of the carboxylic acids according to the present invention are, for example, C1-C8 alkyl, C2-C8 alkenyl, aryl, aralkyl and aralkenyl, hydroxymethyl, C2-C8 hydroxyalkyl, C2-C8 hydroxyalkenyl, aminomethyl, C2-C8 aminoalkyl, cyano, formyl, oxo, thioxo, hydroxy, mercapto, amino, carboxy, or imino groups. Preferred substituents are C1-C8 alkyl, hydroxymethyl, hydroxy, amino, and carboxy groups. Substituents in the a position are particularly preferred. Very particularly preferred substituents are hydroxy, alkoxy, and amino groups, in which context the amino function can be further substituted, if applicable, with alkyl, aryl, aralkyl, and/or alkenyl radicals. Furthermore, the phosphonic and phosphate esters are likewise preferred carboxylic acid derivatives.

Examples of carboxylic acids according to the present invention that may be mentioned are formic acid, acetic acid, propionic acid, butyric acid, isobutyric acid, valeric acid, isovaleric acid, pivalic acid, oxalic acid, malonic acid, succinic acid, glutaric acid, glyceric acid, glyoxylic acid, adipic acid, pimelic acid, suberic acid, azelaic acid, sebacic acid, propiolic acid, crotonic acid, isocrotonic acid, elaidic acid, maleic acid, fumaric acid, muconic acid, citraconic acid, mesaconic acid, camphoric acid, benzoic acid, o,m,p-phthalic acid, naphthoic acid, toluic acid, hydratropic acid, atropic acid, cinnamic acid, isonicotinic acid, nicotinic acid, dicarbamic acid, 4,4′-dicyano-6,6′-dinicotinic acid, 8-carbamoyloctanoic acid, 1,2,4-pentanetricarboxylic acid, 2-pyrrolecarboxylic acid, 1,2,4,6,7-naphthalenepentaacetic acid, malonaldehydic acid, 4-hydroxyphthalamidic acid, 1-pyrazolecarboxylic acid, gallic acid, or propanetricarboxylic acid, a dicarboxylic acid selected from the group that is constituted by compounds having the general formula (N-I), 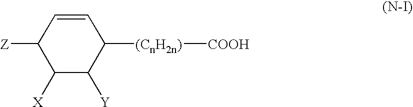
in which Z denotes a linear or branched alkyl or alkenyl group having 4 to 12 carbon atoms, n denotes a number from 4 to 12, and one of the two groups X and Y denotes a COOH group and the other hydrogen or a methyl or ethyl radical, dicarboxylic acids of the general formula (N-I) that additionally carry 1 to 3 methyl or ethyl substituents on the cyclohexene ring, as well as dicarboxylic acids that are obtained from the dicarboxylic acids according to formula (N-I), in formal terms, by addition of a water molecule to the double bond in the cyclohexene ring.

Dicarboxylic acids of formula (N-I) are known in the literature.

A production method may be gathered, for example, from U.S. Pat. No. 3,753,968.

The dicarboxylic acids of formula (N-I) can be produced, for example, by reacting multiply unsaturated dicarboxylic acids with unsaturated monocarboxylic acids in the form of a Diels-Alder cyclization. It is usual to proceed from a multiply unsaturated fatty acid as the dicarboxylic acid component. Linoleic acid, accessible from natural fats and oils, is preferred. Acrylic acid in particular, but also e.g., methacrylic acid and crotonic acid, are preferred as monocarboxylic acid components. Diels-Alder reactions usually yield isomer mixtures in which one component is present in excess. These isomer mixtures, as well as the pure compounds, can be used according to the present invention.

Also usable according to the present invention, in addition to the preferred dicarboxylic acids according to formula (N-I), are those dicarboxylic acids that differ from the compounds according to formula (N-I) by having 1 to 3 methyl or ethyl substituents on the cyclohexyl ring, or are formed from those compounds, in formal terms, by addition of a water molecule to the double bond of the cyclohexene ring.

The dicarboxylic acid (mixture) that is obtained by reacting linoleic acid with acrylic acid has proven particularly advantageous according to the present invention. This is a mixture of 5- and 6-carboxy-4-hexyl-2-cyclohexane-1-octanoic acids. Such compounds are available commercially under the designations Westvaco Diacid® 1550 and Westvaco Diacid® 1595 (manufacturer: Westvaco).

In addition to the short-chain carboxylic acids themselves according to the present invention that have been discussed above by way of example, their physiologically acceptable salts can also be used according to the present invention. Examples of such salts are the alkali, alkaline-earth, zinc salts, and ammonium salts, among which the mono-, di-, and trimethyl-, -ethyl-, and -hydroxyethylammonium salts are also to be understood in the context of the present invention. Acids neutralized with amino acids that react in alkaline fashion, for example arginine, lysine, ornithine, and histidine, can, however, be used in very particularly preferred fashion in the context of the invention. It may furthermore be preferred, for formulation reasons, to select the carboxylic acids from the water-soluble representatives, in particular the water-soluble salts.

It is furthermore preferred according to the present invention to use hydroxycarboxylic acids, and in this context in turn, in particular, the dihydroxy-, trihydroxy-, and polyhydroxycarboxylic acids, as well as the dihydroxy-, trihydroxy-, and polyhydroxydi-, tri-, and polycarboxylic acids. It has been shown in this context that in addition to the hydroxycarboxylic acids, the hydroxycarboxylic acid esters, as well as mixtures of hydroxycarboxylic acids and their esters and also polymeric hydroxycarboxylic acids and their esters, can be used in very particularly preferred fashion. Preferred hydroxycarboxylic acid esters are, for example, full esters of glycolic acid, lactic acid, malic acid, tartaric acid, or citric acid. Further hydroxycarboxylic acid esters that are suitable in principle are esters of β-hydroxypropionic acid, tartronic acid, D-gluconic acid, saccharic acid, mucic acid, or glucuronic acid. Primary linear or branched aliphatic alcohols having 8 to 22 carbon atoms, e.g., fatty alcohols or synthetic fatty alcohols, are suitable as the alcohol components of these esters. The esters of C12-C15 fatty alcohols are particularly preferred in this context. Esters of this type are available commercially, e.g., under the trade name Cosmacol® of EniChem, Augusta Industriale. Particularly preferred polyhydroxypolycarboxylic acids are polylactic acid and polytartaric acid, and their esters.

Also suitable as conditioning ingredients are silicone oils and silicone gums, in particular dialkyl- and alkylarylsiloxanes, for example dimethylpolysiloxane and methylphenylpolysiloxane, as well as their alkoxylated and quaternized analogs. Examples of such silicones are the products marketed by Dow Corning under the designations DC 190, DC 200, and DC 1401, as well as the commercial product Fancorsil® LIM-1.

Likewise suitable as conditioning ingredients according to the present invention are cationic silicone oils, for example the commercially obtainable products Q2-7224 (manufacturer: Dow Corning; a stabilized trimethylsilylamodimethicone), Dow Corning® 939 Emulsion (containing a hydroxylamino-modified silicone that is also referred to as amodimethicone), SM-2059 (manufacturer: General Electric), SLM-55067 (manufacturer: Wacker), and Abil®-Quat 3270 and 3272 (manufacturer: Th. Goldschmidt; diquaternary polydimethylsiloxane, quaternium-80). A suitable anionic silicone oil is the Dow Corning® 1784 product.

The hair treatment agents according to the present invention are subject to no limitations whatsoever with regard to their type of formulation, and can be formulated as an emulsion, creme, solution, gel, or mousse.

Particularly preferred according to the present invention, however, are hair treatment agents that are formulated as an emulsion shampoo.

Further ingredients, adjuvants, and additives are, for example:
thickening agents such as agar-agar, guar gum, alginates, xanthan gum, gum arabic, karaya gum, locust bean flour, linseed gums, dextrans, cellulose derivatives, e.g., methylcellulose, hydroxyalkylcellulose, and carboxymethylcellulose, starch fractions and derivatives such as amylose, amylopectin, and dextrins, clays such as e.g., bentonite, or entirely synthetic hydrocolloids such as polyvinyl alcohol;
hair-conditioning compounds such as phospholipids, for example soy lecithin, egg lecithin, and kephalins;
and silicone oils, perfume oils, dimethyl isosorbide, and cyclodextrins;
solvents and solubilizers such as ethanol, isopropanol, ethylene glycol, propylene glycol, glycerol, and diethylene glycol;
fiber-structure-improving ingredients, in particular mono-, di-, and oligosaccharides, for example glucose, galactose, fructose, fruit sugars, and lactose;
conditioning ingredients such as paraffin oils, vegetable oils, e.g., sunflower oil, orange oil, almond oil, wheat germ oil, and peach-kernel oil, as well as quaternized amines such as methyl-1-alkylamidoethyl-2-alkylimidazolinium methosulfate;
defoaming agents such as silicones;
dyes for coloring the agent;
anti-dandruff ingredients such as piroctone olamine, zinc omadine, and climbazol;
ingredients such as allantoin and bisabolol, cholesterol;
consistency agents such as sugar esters, polyol esters, or polyalkyl ethers;
fats and waxes such as spermaceti, beeswax, montan wax, and paraffins;
fatty acid alkanolamides;
complexing agents such as EDTA, NTA, β-alanine diacetic acid, and phosphonic acids;
swelling and penetrating substances such as primary, secondary, and tertiary phosphates;
opacifiers such as latex, styrene/PVP and styrene/acrylamide copolymers;
luster agents such as ethylene glycol mono- and distearate, and PEG-3 distearate;
pigments;
propellants such as propane-butane mixtures, N₂O, dimethyl ether, CO₂, and air;
antioxidants.

With regard to further optional components as well as the quantities of those components that are used, reference is made explicitly to the relevant manuals known to those skilled in the art, e.g., the monograph of K. H. Schrader, Grundlagen und Rezepturen der Kosmetika [Cosmetic fundamentals and formulations], 2nd ed., Hüthig Buch Verlag Heidelberg, 1989.

A second subject of the invention is cosmetic hair treatment agents containing
a) a surfactant mixture that is made up of
   A) at least one anionic surfactant;
   B) at least one amphoteric surfactant; and
   C) t least one further surfactant selected from the group of the acyl glutamates, the aminoxides, and the alkyl polyglucosides, as well as
b) at least three further hair care substances selected from the group of the
   fatty alcohols;
   cationic surfactants;
   cationic polymers;
   cationically derivatized protein hydrolysates;
   water-insoluble volatile silicones and/or the water-soluble volatile silicones.

A third subject of the invention is cosmetic hair treatment agents containing
a) a surfactant mixture that is made up of
   A) at least one anionic surfactant;
   B) at least one amphoteric surfactant; and
   C) at least one further surfactant selected from the group of the acyl glutamates, the aminoxides, and the alkyl polyglucosides, as well as
b) four further hair care substances, selected each from one component of the groups: fatty alcohols, cationic surfactants, cationic polymers, cationically derivatized protein hydrolysates, water-insoluble or water-soluble volatile silicones, and vitamins and/or provitamins and/or their physiologically acceptable derivatives.

A preferred further hair care substance is, in particular, the combination of cetyl alcohol, behentrimonium chloride, polyquaternium-10, and caprylyl methicone.

A fourth subject of the invention is a method for cleaning and caring for hair, in which an agent according to the present invention is applied onto the hair and is rinsed out again after a contact time.

A fifth subject of the invention is the use of the agent according to the present invention for simultaneous cleaning and conditioning of hair.

The Examples below are intended to explain the subject matter of the invention in more detail without limiting it thereto; all weight indications refer, unless otherwise indicated, to wt %.

### EXAMPLES

| | A | B | C |
|---|---|---|---|
| | wt % | wt % | wt % |
| Sodium laureth sulfate | 10 | 12 | 9 |
| Cocamidopropyl betaine | 4 | 2 | 6 |
| Disodium cocoyl glutamate | 1 | 1 | 2 |
| Cocamidopropyl aminoxide | 1 | | 2 |
| Coco-glucoside | | 2 | |
| Behentrimonium chloride | 1 | 1.5 | 1 |
| Cetyl alcohol | 0.3 | | 0.3 |
| Stearyl alcohol | | 0.7 | 0.3 |
| Polyquaternium-10 | 0.1 | 0.2 | 0.1 |
| Glycol distearate | | | 1 |
| Caprylyl methicone | 0.5 | | 0.4 |
| Laurdimonium hydroxypropyl | | 0.5 | |
| hydrolyzed wheat protein | | | |
| Panthenol | | | 0.3 |
| Preservative | q.s. | q.s. | q.s. |
| Perfume | q.s. | q.s. | q.s. |
| Water | to make 100 | to make 100 | to make 100 |

In hair salon investigations—i.e. application of the product in the so-called half-side test using 2 g of product for the pre-wash and 3 g of product for the main wash, and subsequent assessment by a stylist—the agent according to the present invention exhibited significant advantages in the parameters of wet combability, feel, volume, and weight, as compared with commercially common competitors' products.

The good wet combability of the shampoo according to the present invention was furthermore demonstrated, as compared with commercially common two-in-one shampoos, by biophysical measurements (measurement principle: measuring the force necessary to comb through hair strands; Alkinco 6634 medium-blond strands, measuring 20 strands per sample and averaging).

| | Reduction in wet |
|---|---|
| Product | combability (%) |
| Shampoo according to the present invention | 56.6 |
| Competitor P (two-in-one shampoo) | 44.5 |
| Competitor GF (two-in-one shampoo) | 32.0 |
| Competitor WG (two-in-one shampoo) | 42.9 |
| Competitor HE (two-in-one shampoo) | 20.7 |

The anti-splitting effect was determined using the following method:

Pretreatment of hair strands: The hair strands were pre-cleaned with a 10% alkaline solution of sodium laureth sulfate for 15 minutes in an ultrasonic bath.

Product application: Nine hair strands were treated for each product, plus a control measurement series. This involved wetting the hair strands with tap water and working in 1 g of product per g of hair strand, in each case, After a 5-minute contact time, the hair was rinsed for 90 seconds under cold running tap water. Preconditioning of the hair was performed at 25° C. and a relative humidity of 40% for at least 12 hours.

Experimental method: The hair strands were combed 20,000 times in a climate-controlled chamber (25° C., 40% relative humidity). An ultrafine sieve (mesh size 200 μm) was then used to separate the damaged and undamaged hairs, which were each weighed.

Results (compared with commercially common two-in-one shampoos):

| | Split reduction referred |
|---|---|
| Product | to zero value (%) |
| Shampoo according to the present invention | 88.0 |
| Competitor GF (two-in-one shampoo) | 49.7 |
| Competitor LO (two-in-one shampoo) | 55.6 |
| Competitor JLD (two-in-one shampoo) | 21.8 |

1. A composition comprising
A) at least one anionic surfactant;
B) at least one amphoteric surfactant;
C) at least one further surfactant selected from the group consisting of acyl glutamates, amine oxides and alkyl polyglucosides ; and
D) at least one hair care substance selected from the group consisting of
fatty alcohols;
cationic surfactants;
cationic polymers;
cationically derivatized protein hydrolysates;
water-insoluble volatile silicones and/or the water-soluble volatized silicones; and
vitamins and/or provitamins and/or their physiologically acceptable derivatives.

2. The composition according to claim 1 wherein component D) comprises at least two hair care substances.

3. The composition according to claim 1 further comprising at least one additive selected from the group consisting of emulsifiers, oily substances, plant extracts and short-chain carboxylic acids, salts of short-chain carboxylic acids, silicone oils, cationic silicone oils and silicone gums.

4. The composition according to claim 1 further including at least one of the following ingredients, adjuvants and additives:
thickening agents such as agar-agar, guar gum, alginates, xanthan gum, gum arabic, karaya gum, locust bean flour, linseed gums, dextrans, cellulose derivatives, e.g., methylcellulose, hydroxyalkylcellulose, and carboxymethylcellulose, starch fractions and derivatives such as amylose, amylopectin, and dextrins, clays such as e.g., bentonite, or entirely synthetic hydrocolloids such as polyvinyl alcohol;
hair-conditioning compounds such as phospholipids, for example soy lecithin, egg lecithin, and kephalins;
and silicone oils, perfume oils, dimethyl isosorbide, and cyclodextrins;
solvents and solubilizers such as ethanol, isopropanol, ethylene glycol, propylene glycol, glycerol, and diethylene glycol;
fiber-structure-improving ingredients, in particular mono-, di-, and oligosaccharides, for example glucose, galactose, fructose, fruit sugars, and lactose;
conditioning ingredients such as paraffin oils, vegetable oils, e.g., sunflower oil, orange oil, almond oil, wheat germ oil, and peach-kernel oil, as well as quaternized amines such as methyl-1-alkylamidoethyl-2-alkylimidazolinium methosulfate;
defoaming agents such as silicones;
dyes for coloring the agent;
anti-dandruff ingredients such as piroctone olamine, zinc omadine, and climbazol;
ingredients such as allantoin and bisabolol, cholesterol;
consistency agents such as sugar esters, polyol esters, or polyalkyl ethers;
fats and waxes such as spermaceti, beeswax, montan wax, and paraffins;
fatty acid alkanolamides;
complexing agents such as EDTA, NTA, β-alanine diacetic acid, and phosphonic acids;
swelling and penetrating substances such as primary, secondary, and tertiary phosphates;
opacifiers such as latex, styrene/PVP and styrene/acrylamide copolymers;
luster agents such as ethylene glycol mono- and distearate, and PEG-3 distearate;
pigments;
propellants such as propane-butane mixtures, N₂O, dimethyl ether, CO₂, and air;
antioxidants.

5. A cosmetic hair treatment agent comprising
a) a surfactant mixture that comprises
A) at least one anionic surfactant;
B) at least one amphoteric surfactant; and
C) at least one further surfactant selected from the group consisting of acyl glutamates, amine oxides, and alkyl polyglucosides, and
b) at least two further hair care substances selected from the group consisting of
fatty alcohols;
cationic surfactants;
cationic polymers;
cationically derivatized protein hydrolysates;
water-insoluble volatione silicones and/or the water-soluble volatile silicones; and
vitamins and/or provitamins and/or their physiologically acceptable derivatives.

6. The agent according to claim 5, wherein the anionic surfactant is selected from the group consisting of ethoxylated alkyl sulfates, alkyl sulfates, salts of ether carboxylic acids, and mixtures thereof.

7. The agent according to claim 5, wherein the amphoteric surfactant is selected from the group consisting of alkyl betaines, amidoalkyl betaines, amphoacetates, amphodiacetates, and mixtures thereof.

8. The agent according to claim 5, wherein the surfactant component (c) is an amine oxide.

9. The agent according to claim 5, wherein the surfactant component (C) is either a mixture of acyl glutamates and amine oxides or a mixture of acyl glutamates and polyglucosides.

10. The agent according to claim 5, wherein the ratio of the surfactant components (A):(B):(C): is (3-6):1:(1-2).

11. A cosmetic agent comprising
a) a surfactant that is made up of
A) at least one anionic surfactant;
B) at least one amphoteric surfactant; and
C) at least one further surfactant selected from the group of the acyl glutamates, the amine oxides, and the alkyl polyglucosides, and
b) at least three further hair care substances selected from the group consisting of
fatty alcohols;
cationic surfactants;
cationic polymers;
cationically derivatized protein hydrolysates;
water-insoluble volatile silicones and/or the water-soluble volatile silicones;
vitamins and/or provitamins and/or their physiologically acceptable derivatives.

12. The cosmetic hair treatment agent according to claim 11, wherein the anionic surfactant is selected from the group consisting of ethoxylated alkyl sulfates, alkyl sulfates, or salts of the ether carboxylic acids, and mixtures of these substances.

13. The cosmetic agent according to claim 11, wherein the amphoteric surfactant is selected from the group consisting of alkyl betaines, amidoalkyl betaines, amphoacetates, or amphodiacetates, and from mixtures of these substances.

14. The cosmetic agent according to claim 11, wherein the third surfactant component is an amine oxide.

15. The cosmetic agent according to claim 11, wherein the third surfactant component is either a mixture of acyl glutamates and amine oxides, or a mixture of acyl glutamates and alkyl polyglucosides.

16. The cosmetic agent according to claim 11, wherein the ratio of the surfactant components (A):(B):(C) is (3-6):1:(1-2).

17. The cosmetic hair treatment agent containing
a) a surfactant mixture that is made up of
A) at least one anionic surfactant;
B) at least one amphoteric surfactant; and
C) at least one further surfactant selected from the group of the acyl glutamates, the amine oxides, and the alkyl polyglucosides, as well as
b) four further hair care substances, selected each from one component of the groups: fatty alcohols, cationic surfactants, cationic polymers, cationically derivatized protein hydrolysates, water-insoluble or water-soluble volatile silicones, and vitamins and/or provitamins and/or their physiologically acceptable derivatives.

18. The cosmetic hair treatment agent according to claim 17, wherein the hair care substances b) comprise cetyl alcohol, behentrimonium chloride, polyquaternium-10, and caprylyl methicone.

19. A method for cleaning and caring for hair, wherein an agent according to claim 1 is applied onto the hair and is rinsed out again after a contact time.

20. A method of the simultaneous cleaning and conditioning of hair, said method comprising the step of applying to the hair the agent of claim 1.
